(19) United States
(12) Patent Application Publication
(10) Pub. No.: US 2014/0243807 A1
(43) Pub. Date: Aug. 28, 2014

(54) DEVICES AND METHODS FOR DENERVATION OF THE NERVES SURROUNDING THE PULMONARY VEINS FOR TREATMENT OF ATRIAL FIBRILLATION

(71) Applicant: James Margolis, Coral Gables, FL (US)

(72) Inventor: James Margolis, Coral Gables, FL (US)

(21) Appl. No.: 14/186,323

(22) Filed: Feb. 21, 2014

Related U.S. Application Data
(60) Provisional application No. 61/768,151, filed on Feb. 22, 2013.

(51) Int. Cl. A61B 18/14; A61B 18/02

(52) U.S. Cl. CPC A61B 18/1492; A61B 18/0206; A61B 2018/0212
USPC 606/20; 606/41; 606/34

(57) ABSTRACT

Methods, systems, and devices for providing a denervating energy treatment to the tissue of the pulmonary vein utilizing a catheter-based structure having one or more energy delivery surfaces. In some instances energy delivery surfaces are arranged with a circumferential and axial offset relative to one another. A pattern of individual lesions loosely approximating a helix are placed at the pulmonary vein so as to provide a pattern which covers substantially the circumference of the pulmonary vein with an axial offset that distributes the lesions to avoid stenosis. Denervating energy is applied by modulation of the energy delivery surfaces using an energy source integrated with a controller and control algorithm. In some instances feedback is used in a control algorithm for energy modulation. Energy sources are radiofrequency, ultrasound, and cryogenic.

### CROSS REFERENCE TO RELATED APPLICATIONS

This application claims the benefit of U.S. Provisional Application 61/768,151 Devices and Methods for Denervation of the Nerves Surrounding the Pulmonary Veins for Treatment of Atrial Fibrillation, filed Feb. 22, 2013.

### STATEMENT REGARDING FEDERALLY FUNDED RESEARCH OR DEVELOPMENT

None.

### BACKGROUND

Atrial fibrillation (“AF”) is the most common cardiac arrhythmia causing the muscles of the atria to contract in an irregular quivering motion rather than in the coordinated contraction that occurs during normal cardiac rhythm. AF may be detected by the presence of an irregular pulse or by the absence of p-waves on an electrocardiogram. During an episode of AF, the regular electrical impulses that are normally generated by the sinoatrial (SA) node are overwhelmed by rapid disorganized electrical impulses in the atria. These disorganized impulses are induced by “triggers” that are usually, though not always, located in and around the orifices of the pulmonary veins. Because the resultant disorganized impulses of AF reach the atrioventricular (AV) node in a rapid (up to 600 per minute) and highly irregular manner, the impulses that are subsequently filtered and conducted through the AV node to the ventricles are also rapid (around 150 per minute). AF episodes may be intermittent (“paroxysmal”) lasting from seconds to weeks or they may last for years, in which case the AF may be referred to as “chronic AF”. Untreated paroxysmal AF ususally leads to chronic AF.

Although patients do not usually experience immediate life-threatening problems from the onset of AF, they commonly experience immediate symptoms such as palpitations of the heart, weakness, tiredness, and shortness of breath. The most serious complication of AF is the risk of stroke caused by the pooling and stasis of blood in the left atrial appendage (LAA) that results in the formation of clots that may break off and travel to the brain. Patients with chronic AF lose up to 20% of their pumping capacity. This leads to chronic fatigue and even heart failure.

Paroxysmal atrial fibrillation may be treated by ablation of nerve fibers surrounding the pulmonary veins. Ablation of these nerve fibers has been demonstrated to prevent AF triggering events, and in many cases may cure the problem. The procedure used to produce ablation patterns that isolate and prevent AF triggering events requires lengthy, invasive medical procedures that require a very high degree of specialized medical skill, and which are frequently ineffective and not always permanent.

A myriad of devices using various forms of energy (RF devices, ultrasound and cryothermia) have been tried to simplify the procedure, to increase the completeness of ablation, and consequently the success rate. Confounding all of the devices is the trade-off between completeness of denervation and procedural complications—phrenic nerve palsy, esophageal rupture and pulmonary vein stenosis. The right phrenic nerve courses close to the right superior pulmonary vein (J Cardiovasc Electrophysiol. 2005 March; 16(3):309-13; the contents of which being incorporated herein by reference in their entirety), and the esophagus nearly abuts the left atrium (Circulation. 2005; 112:1400-1405; the contents of which being incorporated herein in their entirety). Both structures have courses varying from individual to individual. Imprecision in ablation in areas close to the phrenic nerve or the esophagus can lead to devastating and sometimes fatal complications.

For maximum effectiveness, pulmonary vein ablation should address all fibers in a circumferential manner. However, circumferential application of RF energy using presently available technology can lead to pulmonary vein stenosis—a serious and basically untreatable complication. It has been shown that the body's natural response to the placement of an ablation lesion is a localized proliferation of smooth muscle cells that may increase the thickness of the tissue, and hence, reduce the open area of blood flow in the context of the circulatory system (a “stenosis”). Ablation lesions that are too concentrated present a risk of causing a concentrated proliferation of tissue resulting in stenosis. Too obviate this problem ablations are performed as a series of ablation points around the pulmonary veins and the pulmonary venous antra in an effort to create a pattern of lesions sufficient to cover the circumference of the vessel but diffuse enough in arrangement to prevent a stenosis. This naturally creates a balancing of the risks between incomplete lesion coverage, which may result in further AF episodes, and, an overly dense lesion coverage which may result in any of stenosis, phrenic nerve damage, and esophageal damage. With available tools and methods, the procedure is both imprecise and time consuming, and the procedure demands a high degree of medical skill to perform. With presently used methods of RF ablation, which use imprecise temperature control charring of blood and tissue is an additional problem. Because of the procedure length, adequate anticoagulation is difficult to maintain throughout the procedure. Both charring and blood clots can lead to stroke (both overt and silent) as a not uncommon complication of this procedure. Incidence is approximately 1%, with silent strokes detectable by advanced MRI techniques occurring 25-30% of case. A shortened procedure that helps to avoid negative side effects is needed.

### Similarity of Problem of Pulmonary Vein Denervation and Renal Denervation

It has been conclusively demonstrated that the nerves surrounding the renal arteries can be effectively denervated with heat energy applied either with RF energy or ultrasound applied from within the lumen; and when this energy is appropriately regulated, denervation can occur without damage to the renal artery. The distribution of the nerves surrounding the pulmonary veins and those surrounding the renal arteries is similar, as is the distance of the nerves from the vessel lumen. Two dissimilarities between the systems are vessel diameters (4-8 mm for renal arteries and 9-13 mm for pulmonary veins), and differing characteristics of the vessel walls. The fact that transmission of heat energy through renal arterial walls can be done without damage to the artery does not assure that pulmonary veins would not be so damaged during transmission of the same energy. However, there is a large human experience delivering RF energy at high doses to the pulmonary veins. By using energy below a charring or vaporizing temperature, in the manner described above, damage to the veins may be avoided unless the energy is delivered in a circumferentially concentrated manner. Therefore, learning from treatment methods and devices that have been successfully applied in the renal arteries is useful in providing improvements to methods and devices available to address the complex problems associated with AF treatments.

In light of the foregoing, there remains a need to provide a simple device and method of treatment to produce pulmonary vein lesions sufficient to halt AF episodes while avoiding the complications caused by tissue trauma or incomplete pulmonary vein isolation.

### SUMMARY OF THE INVENTION

The isolation of pulmonary veins in an AF treatment presents a complex set of problems for the physician. The very close proximity of the phrenic nerve and esophagus to the pulmonary veins requires that treatment energy be carefully and precisely delivered so as to avoid affecting tissues collateral to the pulmonary veins. However, a less than complete delivery of treatment energy can leave open a conductive path for the underlying stray electrical currents that cause AF. Coupled with these two problems is the additional problem that a stenosis may develop in response to an overly concentrated grouping of lesions in the pulmonary veins. The best solution to this set of problems is to deliver treatment energy in a pattern that covers substantially the full circumference of the pulmonary vein lumen where the pattern is a plurality of individual lesions that are circumferentially and axially offset so as to form a pattern that loosely approximates a helix. Such a pattern axially distributes the treatment lesions such that if some stenosis were to naturally occur in response to treatment, the overall effect of stenosis is dispersed enough to avoid a deleterious reduction of the cross section of the pulmonary vein lumen at any given point along its length.

A catheter-based expandable structure with a plurality of energy delivery surfaces is a particularly advantageous way to access the pulmonary veins. Structures with a circumferentially and axially offset array of energy delivery surfaces may be made suitable for pulmonary vein isolation by sizing structures from approximately 8 mm to approximately 16 mm and arranging the number and location of energy delivery surfaces so as to provide the loosely helical lesion pattern that covers the full circumference of the inner pulmonary vein lumen while providing an axial dispersion sufficient to prevent a deleterious reduction in lumen diameter from stenosis. In many embodiments, the lesion pattern is created at a plurality of locations simultaneously during the delivery of treatment energy.

Examples of suitable catheter-based structures that may be modified to perform pulmonary denervating vein isolation in accordance with the present invention include those shown in U.S. patent application Ser. Nos. 12/206,591; 10/232,909; 11/420,419; 13/087,163; 11/782,451; 10/938,138; 11/392,231; 12/640,664; 11/420,712; 12/616,758; 13/087,163; 11/782,451; 12/700,524; 11/975,651; 11/975,474; 12/127,287; 13/562,150, the complete contents of each being incorporated herein by reference.

Another example of a balloon structure is from Vessix Vascular of Laguna Hills, Calif., which has been publicly disclosed on its website (www.vessixvascular con)) and at medical conferences, wherein a balloon catheter has surface mounted flexible circuit electrodes that deliver bipolar radiofrequency energy. The Vessix Vascular balloon catheter design can be adapted to provide a denervating pulmonary vein isolation treatment of the present invention.

Additionally, other structures such as expandable coils or probes common to current AF treatment procedures may be adapted to provide a denervating energy to isolate the pulmonary veins as described by the present invention.

To achieve complete isolation of the pulmonary vein through a denervation energy treatment of the present invention, a plurality of one or more surfaces is used; the maximum number of energy delivery surfaces may be limited by size and stiffness constraints of the catheter which would be associated with the quantity of energy conductors leading from the energy delivery surfaces to the energy source. The size and spacing of energy delivery surfaces is arranged based on the desired size of the lesion created by the treatment energy dose. The denervating energy doses of the present invention are lower than the tissue vaporizing or burning energy doses of prior AF treatment approaches. Therefore a larger energy delivery surface may be employed. However, the optimized sizing of the surface is ultimately a function of the power of the energy delivered. In radiofrequency (“RF”) energy embodiments the spacing between energy delivery surfaces may range from about 0.1 mm to about 20 mm.

Structures with energy delivery surfaces may be further comprised to include one or more temperature sensing devices such as thermistors or thermocouples mounted in proximity to one or more energy delivery surfaces. Temperature sensing devices may be configured to provide feedback information to a control algorithm in a controller adapted to operate in conjunction with an energy source. Denervating pulmonary vein isolation provides an energy treatment sufficient to denature tissues without causing vaporization or charring of tissue. Therefore, a temperature-based control algorithm is a preferred method for maintaining treatment temperatures below that which would vaporize or char tissue. In addition, one or more of voltage, current, and impedance may be used as primary or secondary control algorithm factors. Embodiments of the present invention use one or more of temperature, voltage, current, and impedance as control algorithm factors to deliver energy sufficient to cause pulmonary vein isolation by denervating tissue without causing vaporization or charring. At any point before or during the application of treatment energy, an energy delivery surface may be temporarily modulated or completely deactivated if a feedback condition is outside of algorithm parameters. This approach helps to avoid the risk of coagulum formation, and phrenic nerve or esophageal damage, while providing energy sufficient to denature nerve tissue, and hence, isolate the pulmonary veins to achieve an efficacious treatment of AF while avoiding the risk of stenosis.

The temperature to achieve denervation is approximately 50C to approximately 80C with a treatment energy of approximately 0.25 W to approximately 100 W and with a treatment duration of approximately 10 seconds to approximately 5 minutes.

The method of access to the pulmonary veins may be by any of those used in AF treatment and catheter-based intervention including endoscopically through the wall of the heart, by a percutaneous venous approach, or by an arterial approach.

In one preferred embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of individual flexible circuit electrodes positioned with a circumferential and axial offset from one another so as to loosely approximate a helical pattern on the surface of the balloon. The electrodes are configured to deliver bipolar RF energy. Conductors passing through the body of the catheter electrically connect the electrodes to a RF generator and controller. The electrodes are individually configured to be energized and controlled in a modulated fashion so as to precisely maintain a treatment temperature in accordance with a control algorithm. Temperature may or may not be ramped according to the treatment algorithm. The treatment energy is applied in accordance with the treatment algorithm and a denervating energy treatment is delivered to accomplish isolation of the pulmonary vein as part of an AF treatment procedure.

In another embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of individual flexible circuit electrodes positioned with a circumferential and axial offset from one another so as to loosely approximate a helical pattern on the surface of the balloon. The electrodes are configured to deliver monopolar RF energy. A common ground may be one of the electrodes, which in turn may optionally be varied by the control algorithm so as to select different electrodes as the ground during the course of treatment, or an external grounding pad may be employed. Conductors passing through the body of the catheter electrically connect the electrodes to a RF generator and controller. The electrodes are optionally individually configured to be energized and controlled in a modulated fashion to maintain a treatment temperature in accordance with a control algorithm. The treatment energy is applied in accordance with the treatment algorithm and a denervating energy treatment is delivered to accomplish isolation of the pulmonary vein as part of an AF treatment procedure.

In another embodiment of the present invention, the catheter-based expandable structure is configured to include RF electrodes on a basket-like structure, which may be closed or open-ended at the basket-like structure's distal end.

In another embodiment of the present invention, the catheter-based expandable structure configured to include RF electrodes is a coil-like structure, which includes electrodes at points along the coil, which are positioned to create a series of energy delivery locations that loosely approximate a helical pattern as described herein.

In another embodiment of the present invention, the catheter-based device is configured to include a probe-like structure wherein the probe is configured to include one or more RF electrodes at its distal end.

In another embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of ultrasound transducers positioned with a circumferential and axial offset from one another so as to loosely approximate a helical pattern on the surface of the balloon. Conductors passing through the body of the catheter connect the ultrasound transducers to a generator and controller. The ultrasound transducers are optionally individually configured to be energized and controlled in a modulated fashion to maintain a treatment temperature in accordance with a control algorithm. The treatment energy is applied in accordance with the treatment algorithm and a denervating energy treatment is delivered to accomplish isolation of the pulmonary vein as part of an AF treatment procedure.

In another embodiment of the present invention, the catheter-based expandable structure is configured to include ultrasound transducers on a balloon, which is part of a catheter system having an ultrasound energy source and controller.

In another embodiment of the present invention, the catheter-based expandable structure is configured to include ultrasound transducers on a basket-like structure, which may be closed or open-ended at the basket-like structure's distal end, and which is part of a catheter system having an ultrasound energy source and controller.

In another embodiment of the present invention, the catheter-based expandable structure is configured to include ultrasound transducers on a coil structure, which is part of a catheter system having an ultrasound energy source and controller.

In another embodiment of the present invention, the catheter-based structure is configured to include ultrasound transducers on a probe-like structure, which is part of a catheter system having an ultrasound energy source and controller.

In another embodiment of the present invention, a cryogenic energy source is operatively coupled to the energy delivery surfaces of any of the structures described herein. The delivery of the cryogen is modulated to the energy delivery surfaces according to a control algorithm and feedback as described herein so as to create an approximately helical pattern of PV-isolating lesions adjacent the energy delivery surfaces while avoiding damage to tissues such as the phrenic nerve and esophagus.

### BRIEF DESCRIPTION OF THE DRAWINGS

FIG. 1 shows a schematic view of the human heart with an example device embodiment accessing a pulmonary vein.

FIG. 2 shows a schematic view of a balloon catheter device embodiment used in the present invention.

FIG. 3 shows a representative lesion pattern embodiment of the present invention.

FIGS. 3A and 3B show the lesion pattern of FIG. 3 unrolled about axis a-b in a flat plane, and a sectional view about plane X-X.

FIG. 4 shows a schematic sectional view of a lesion pattern and an example device embodiment of the present invention.

FIG. 4A shows a sectional view of a lesion pattern embodiment of the present invention as viewed in a circumferential cross section at a location distal from the lesion pattern looking proximally toward the pulmonary vein ostium.

FIG. 5 shows a schematic view of a closed basket structure embodiment at the distal end of a catheter device used in the present invention.

FIG. 5A shows a schematic view of an open basket structure embodiment at the distal end of a catheter device used in the present invention.

FIG. 6 shows a schematic view of a coil structure embodiment at the distal end of a catheter device used in the present invention.

FIG. 7 shows a schematic view of a probe structure embodiment at the distal end of a catheter device used in the present invention.

FIG. 8 shows a schematic view of an energy delivering catheter system embodiment used in the present invention.

FIG. 9 shows the steps of a treatment method embodiment of the present invention.

FIG. 10 shows a schematic view of a cryogenic balloon embodiment of the present invention.

### DETAILED DESCRIPTION OF THE INVENTION

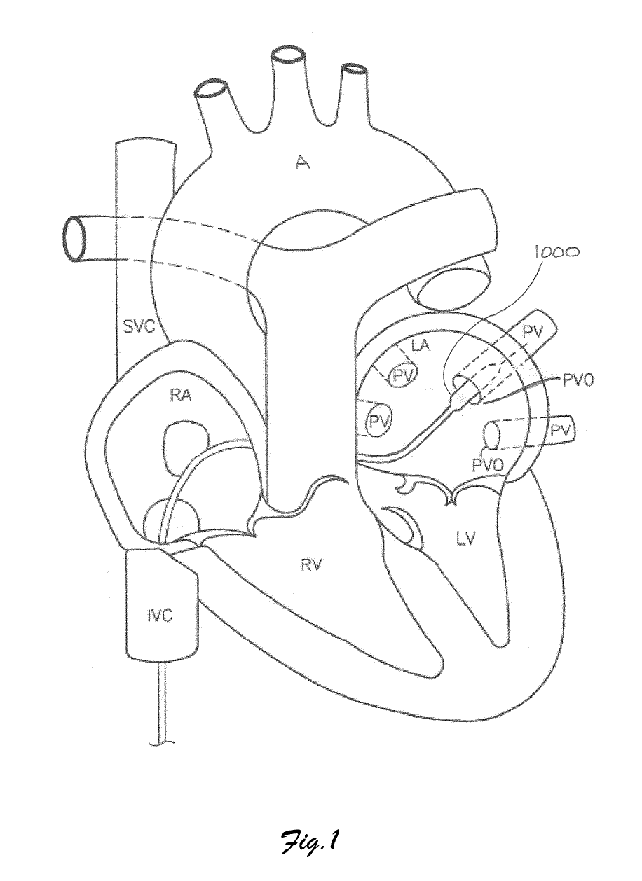

Referring to FIG. 1, the human heart is a complex hollow structure having numerous discrete sub-structures. The four chambers of the heart are the right atrium (“RA”), the right ventricle (“RV”), the left atrium (“LA”), and the left ventricle (“LV”). Several major blood vessels flow to or from the heart. The inferior and superior vena cava (“IVC” and “SVC” respectively) return blood to the heart. The aorta (“A”) supplies blood to the major portion of the body from the heart. The pulmonary veins (“PV”) provide blood from the lungs to the heart. Inside the LA are the four openings where blood from the lungs enter the LA from the PV through the pulmonary venous ostia (“PVO”). Shown is an exemplary embodiment of a balloon catheter device 1000 for use in the present invention. A venous approach to the heart through the IVC is shown, However, any of the large variety of interventional access methods used for heart procedures may be used. For example, arterial access may be used, endoscopic access may be used by a method such as by transapical or subxyphoid approach, and the like, depending on the preferences of the physician performing the AF treatment. The choice of approach may be influenced in part by the device embodiment used to create the lesion pattern of the present invention.

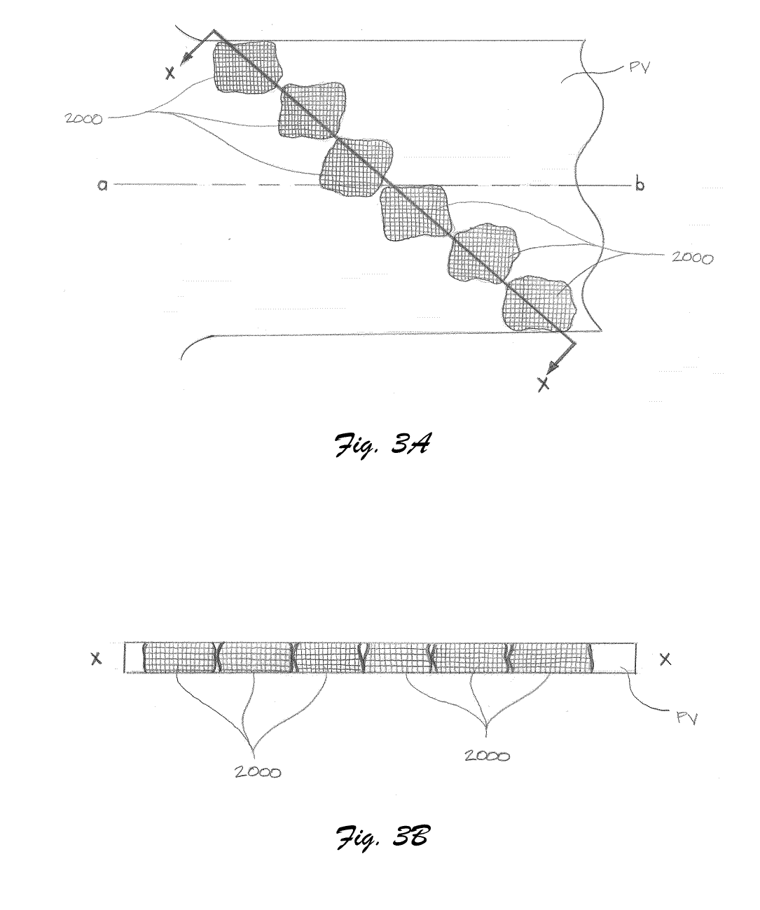

Referring to FIGS. 3, 3A, 3B, a pattern of lesions 2000 is created about the circumference and length of PV such that when viewed in a flat plane (FIG. 3A) or in a plane perpendicular to the lesion pattern (FIG. 3B), a substantially continuous pattern of lesions 2000 is formed about the circumference of PV where each of the lesions 2000 is axially offset from one another along the length of the PV.

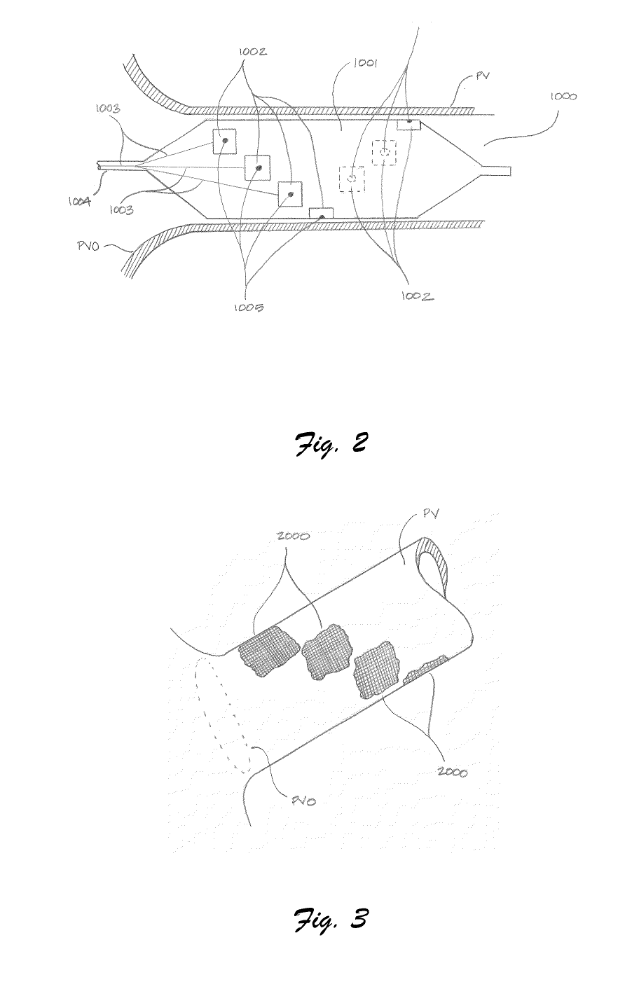

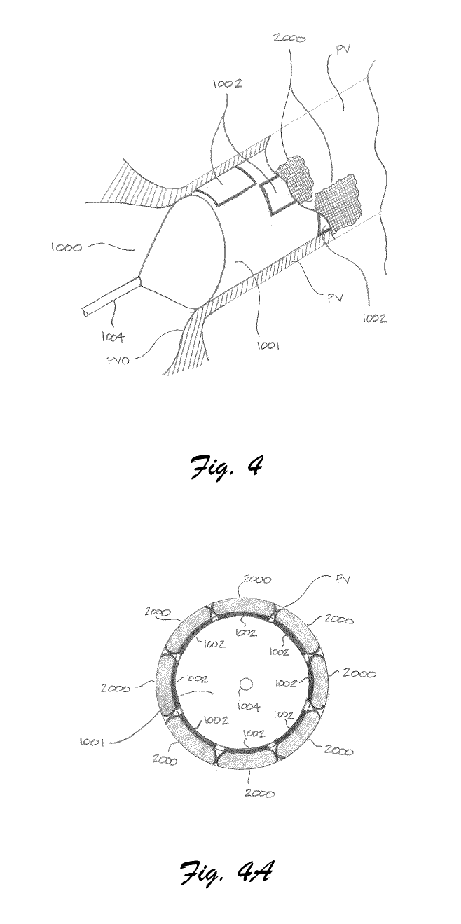

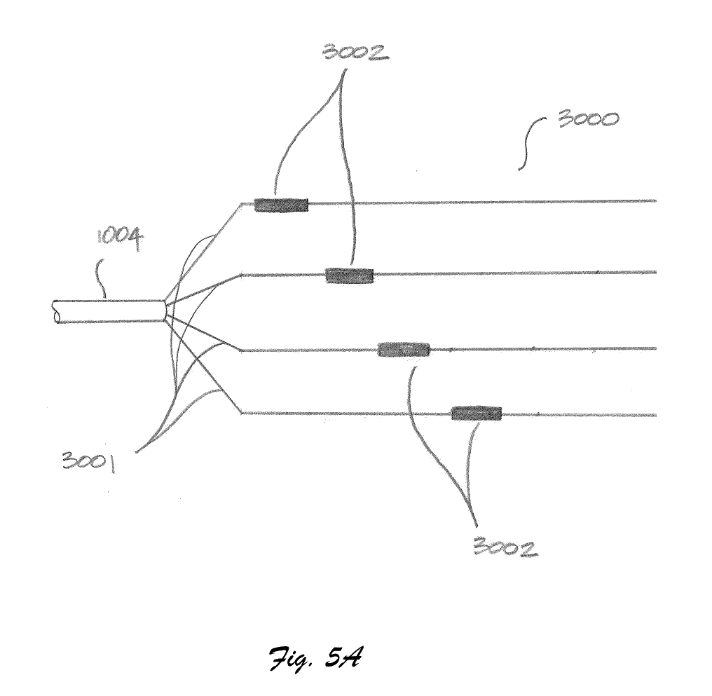

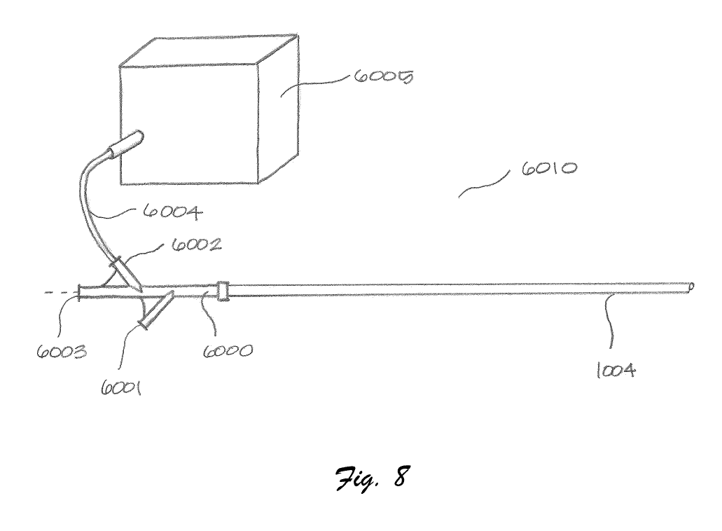

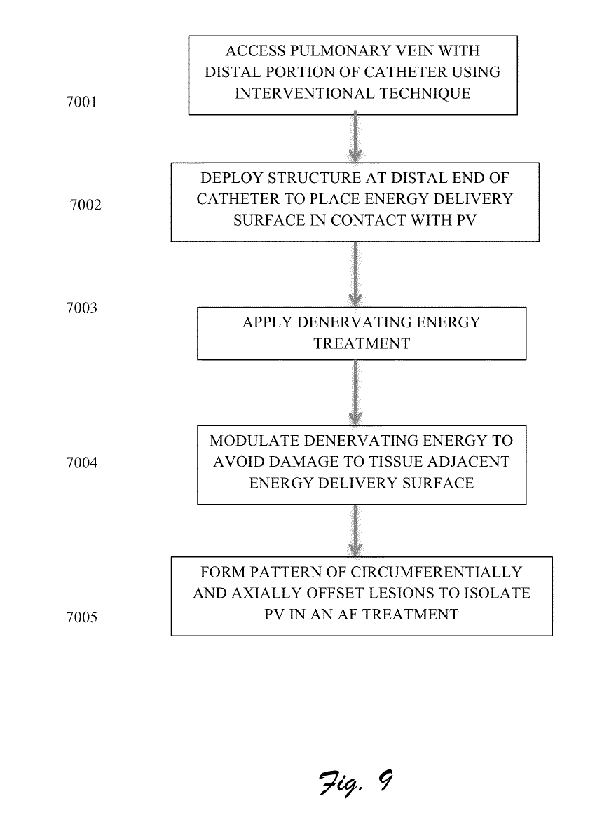

Referring now to FIG. 2, a balloon catheter device 1000 is shown having been positioned and inflated just at the PVO and inside the PV. Balloons may range in expanded diameter from about 8 mm to about 16 mm, which may further include tapered diameters with the larger diameter being at the ostial end of the balloon when placed in the PV. A plurality of energy delivery surfaces 1002 are positioned to be circumferentially and axially offset from one another on balloon 1001 so as to loosely approximate a helical pattern. Any circumferentially and axially staggered pattern may be used, the term helical being a convenient description for any staggered pattern employed. Adjacent or integrated with energy surfaces 1002, one or more optional temperature sensors 1005 may be included. Temperature sensors 1005 may be thermistors or thermocouples and may be in direct or indirect contact with tissue and/or the energy delivery surfaces 1002. Conductors 1003 run proximally through catheter body 1004 and operatively connect the energy delivery surfaces to an energy source and controller. FIG. 8 shows a catheter system 6010 with an integrated energy source and controller 6005. Catheter body 1004 (as also shown in FIGS. 2, 4, 4A, 5, 5A, 6, 7, and 8) is operatively connected to power source 6005 by a connector 6004 such that conductors pass through a port 6002 of a catheter hub 6000. Catheter hub 6000 may have a guidewire and/or fluid conducting port 6003 in communication with a lumens in catheter body 1004. Catheter hub 6000 may have an inflation port 6001 in communication with lumens in catheter body 1004. The configurations of ports in catheter hub 6000 and lumens in catheter body 1004 may depend on the structural embodiment at the distal end of the catheter where the energy surfaces are located. For example, catheter body 1004 would have an inflation lumen for embodiments where a balloon is located at its distal end, while baskets, coils and probes would not require an inflation lumen but may be configured to include a lumen for guidewires. aspiration and/or perfusion. Additionally, baskets, coils or probes may include mechanical devices for deployment and/or tip deflections. A guidewire lumen would be a preferred embodiment of catheter body 1004 given that over-the-wire and rapid exchange configurations are standard in catheter-based interventional tools.

Referring to FIGS. 1-4A, and FIG. 8, a balloon catheter system 6010 with distal configuration 1000, is positioned into the PV such that the proximal end of balloon 1001 is just inside the PVO. Balloon 1001 is deployed and expanded to place it in contact with the lumen of the PV.

In some embodiments, on balloon 1001 is an array of energy delivery surfaces 1002 configured as individual flexible circuit electrodes positioned with a circumferential and axial offset from one another so as to loosely approximate a helical pattern on the surface of balloon 1001. The electrodes 1002 are configured to deliver bipolar RF energy. Conductors 1003 passing through catheter body 1004 electrically connect the electrodes 1002 to a RF generator and controller 6005 via a catheter hub 6000 and an electrical connector 6004. The electrodes 1002 are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions 2000 corresponding to the position of the electrodes 1002. The resultant pattern of lesions 2000 is distributed at point locations about the circumference and length of PV, and when viewed in a plane perpendicular to the length of PV cover substantially the complete circumference of PV.

The denervating energy treatment is applied in the form of a mild heating of tissue which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50C to about 80C. In this range, the conductive nerve tissue in the wall of the PV undergoes cellular necrosis while avoiding the gross tissue trauma, and resultant cellular proliferation, that results from vaporization or charring.

The control algorithm for generator 6005 may detect contact with tissue by sensing impedance levels at electrodes 1002. The algorithm selectively energizes electrodes 1002 when the treatment is initiated. Individual control of electrodes 1002 may be accomplished by modulating a time and/or level of powering in accordance with the control algorithm and feedback sensed at the electrodes 1002 and/or temperature sensors 1005. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode 1002 is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 9 mm to about 13 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids charring, stenosis, phrenic nerve damage, or esophageal damage.

Referring again to FIGS. 1-4A, and FIG. 8, another embodiment of the present invention, energy delivery surfaces 1002 are electrodes configured to deliver monopolar RF energy. A common ground may be one of the electrodes 1002, which in turn may optionally be varied by the control algorithm so as to select different electrodes 1002 as the ground during cycle time periods over the course of treatment, or an external grounding pad (not shown) may be employed. Conductors 1003 passing through catheter body 1004 electrically connect the electrodes 1002 to a RF generator and controller 6005 via a catheter hub 6000 and an electrical connector 6004. The electrodes 1002 are optionally individually configured to be energized and controlled in a modulated fashion to maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions 2000 corresponding to the position of the electrodes 1002. The resultant pattern of lesions 2000 is distributed at point locations about the circumference and length of PV, and when viewed in a plane perpendicular to the length of PV cover substantially the complete circumference of PV. The application of monopolar RF energy during the course of a treatment ranges from approximately 0.25 W to approximately 100 W of power for a total treatment time of up to approximately 5 minutes.

In another embodiment, the energy delivery surfaces 1002 on balloon 1001 are an array of ultrasound transducers. Ultrasound transducers 1002 are optionally individually configured to be energized and controlled in a modulated fashion to maintain a treatment temperature in accordance with a control algorithm. The ultrasound transducers 1002 may produce focused or unfocused ultrasound.

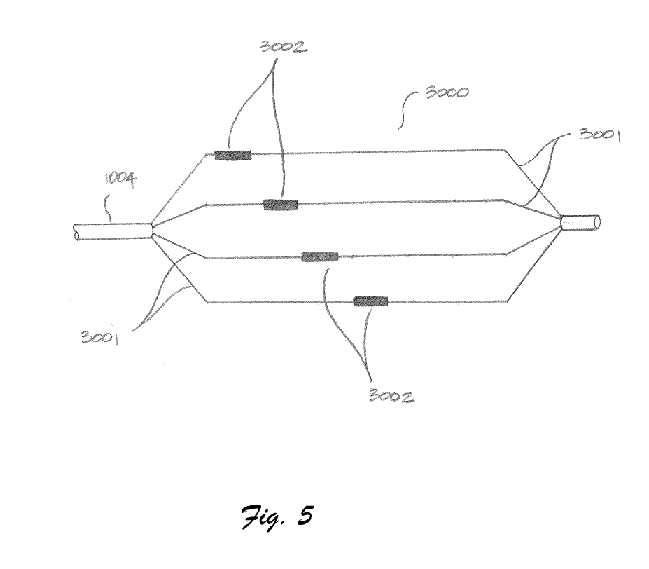

Referring to FIGS. 1, 3, 4A, 5, 5A, and 8, the catheter-based system 6010 is configured with a basket-like expandable structure 3000 at the distal end of catheter body 1004 which may range in expanded diameter from about 8 mm to about 16 mm. Optionally, basket structure 3000 may be open on its distal end as shown in FIG. 5A. Expandable structure 3000 has a plurality of struts 3001 that expand when deployed either by mechanical means such as a pull wire or by making struts 3001 from a shape memory material such as nickel-titanium. Mounted on struts 3001 is an array of energy delivery surfaces 3002,

In some embodiments, energy delivery surfaces 3002 are configured as individual flexible electrodes positioned with a circumferential and axial offset from one another so as to loosely approximate a helical pattern on the surface of basket structure 3000. Adjacent or integrated with energy surfaces 3002, one or more optional temperature sensors may be included. Temperature sensors may be thermistors or thermocouples and may be in direct or indirect contact with tissue and/or the energy delivery surfaces 3002.

In some embodiments, the electrodes 3002 are configured to deliver bipolar RF energy. Conductors (not shown) passing through catheter body 1004 electrically connect the electrodes 3002 to a RF generator and controller 6005 via a catheter hub 6000 and an electrical connector 6004. The electrodes 3002 are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions 2000 corresponding to the position of the electrodes 3002. The resultant pattern of lesions 2000 is distributed at point locations about the circumference and length of PV, and when viewed in a plane perpendicular to the length of PV cover substantially the complete circumference of PV.

The denervating energy treatment is applied in the form of a mild heating of tissue which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50C to about 80C. In this range, the conductive nerve tissue in the wall of the PV undergoes cellular necrosis while avoiding the gross tissue trauma, and resultant cellular proliferation that results, from vaporization or charring.

The control algorithm for generator 6005 may detect contact with tissue by sensing impedance levels at electrodes 3002. The algorithm selectively energizes electrodes 3002 when the treatment is initiated. Individual control of electrodes 3002 may be accomplished by modulating a time and/or level of power in accordance with the control algorithm and feedback sensed at the electrodes 3002 and/or temperature sensors. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode 3002 is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 9 mm to about 13 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids stenosis, phrenic nerve damage, or esophageal damage.

Alternately, energy delivery surfaces 3002 may be configured to be electrodes delivering monopolar RF energy. A common ground may be one of the electrodes 3002, which in turn may optionally be varied by the control algorithm so as to select different electrodes 3002 as the ground during cycle time periods over the course of treatment, or an external grounding pad (not shown) may be employed. Conductors (not shown) passing through catheter body 1004 electrically connect the electrodes 3002 to a RF generator and controller 6005 via a catheter hub 6000 and an electrical connector 6004. The electrodes 3002 are optionally individually configured to be energized and controlled in a modulated fashion to maintain a treatment temperature in accordance with a control algorithm programmed in the software memory of controller 6005.

In an additional monopolar electrode configuration, the struts 3001 may themselves be conductive and areas adjacent to electrode 3002 surfaces on struts 3001 are insulated from conducting energy to tissue of the PV.

The application of monopolar RF energy during the course of a treatment ranges from approximately 0.25 W to approximately 100 W of power for a total treatment time of up to approximately 5 minutes.

In another embodiment, the energy delivery surfaces 3002 on struts 3001 are an array of ultrasound transducers. Ultrasound transducers 3002 are optionally individually configured to be energized and controlled in a modulated fashion to maintain a treatment temperature in accordance with a control algorithm. The ultrasound transducers 3002 may produce focused or unfocused ultrasound.

Referring now to FIGS. 1, 3, 4A, 6, and 8, in an embodiment of the present invention, the catheter-based system 6010 is configured with a coil-like expandable structure 4000 at the distal end of catheter body 1004 ranging in expanded diameter from about 8 mm to about 16 mm, which includes energy delivery surfaces 4002 at points along the body 4001 of the coil, and which are positioned to create a series of energy delivery locations that loosely approximate a helical pattern as described herein. Adjacent or integrated with energy surfaces 4002, one or more optional temperature sensors may be included. Temperature sensors may be thermistors or thermocouples and may be in direct or indirect contact with tissue and/or the energy delivery surfaces 4002.

In some embodiments, the energy delivery surfaces 4002 are electrodes configured to deliver bipolar RF energy. Conductors (not shown) passing through catheter body 1004 electrically connect the electrodes 4002 to a RF generator and controller 6005 via a catheter hub 6000 and an electrical connector 6004. The electrodes 4002 are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions 2000 corresponding to the position of the electrodes 4002. The resultant pattern of lesions 2000 is distributed at point locations about the circumference and length of PV, and when viewed in a plane perpendicular to the length of PV cover substantially the complete circumference of PV.

The control algorithm for generator 6005 may detect contact with tissue by sensing impedance levels at electrodes 4002. The algorithm selectively energizes electrodes 4002 when the treatment is initiated. Individual control of electrodes 4002 may be accomplished by modulating a time and/or level of powering in accordance with the control algorithm and feedback sensed at the electrodes 4002 and/or temperature sensors. The algorithm may use any of temperature, voltage, current, and impedance as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode 4002 is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 9 mm to about 13 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids stenosis, phrenic nerve damage, or esophageal damage.

Alternately, energy delivery surfaces 4002 may be electrodes configured to deliver monopolar RF energy. A common ground may be one of the electrodes 4002, which in turn may optionally be varied by the control algorithm so as to select different electrodes 4002 as the ground during cycle time periods over the course of treatment, or an external grounding pad (not shown) may be employed. Conductors (not shown) passing through catheter body 1004 electrically connect the electrodes 4002 to a RF generator and controller 6005 via a catheter hub 6000 and an electrical connector 6004. The electrodes 4002 are optionally individually configured to be energized and controlled in a modulated fashion to maintain a treatment temperature in accordance with a control algorithm programmed in the software memory of controller 6005.

In an additional monopolar electrode configuration, the coil body 4001 may itself be conductive and the spaces between electrode 4002 surfaces on coil body 4001 are insulated from conducting energy to tissue of the PV.

The application of monopolar RF energy during the course of a treatment ranges from approximately 0.25 W to approximately 100 W of power for a total treatment time of up to approximately 5 minutes.

In another embodiment, the energy delivery surfaces 4002 on coil body 4001 are an array of ultrasound transducers. Ultrasound transducers 4002 are optionally individually configured to be energized and controlled in a modulated fashion to maintain a treatment temperature in accordance with a control algorithm. The ultrasound transducers 4002 may produce focused or unfocused ultrasound.

Referring now to FIGS. 1, 3, 4A, 7, and 8, in an embodiment of the present invention, the catheter-based system 6010 is configured with a steerable probe-like expandable structure 5000 at the distal end of catheter body 1004, which includes energy delivery surface 5002 at points along the body 5001 of the probe. Probe body 5001 may be deflected via a control wire (not shown) to deflect the probe body 5001 and energy delivery surface 5002 to any angle up to approximately 90 degrees from the undeflected position. Adjacent or integrated with energy delivery surface 5002, an optional temperature sensor may be included. The temperature sensor may be a thermistor or a thermocouple and may be in direct or indirect contact with tissue and/or the energy delivery surface 5002. The energy delivery surface 5002 is an electrode configured to deliver bipolar RF energy. Conductors (not shown) passing through catheter body 1004 electrically connect the electrode 5002 to a RF generator and controller 6005 via a catheter hub 6000 and an electrical connector 6004. The electrode 5002 is configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating in series a pattern of lesions 2000. The resultant pattern of lesions 2000 is distributed at point locations about the circumference and length of PV, and when viewed in a plane perpendicular to the length of PV cover substantially the complete circumference of PV.

Alternately, electrode 5002 may be configured to deliver monopolar RF energy. A ground may be located on probe body 5001 proximal to electrode 5002, or an external grounding pad (not shown) may be employed. Conductors (not shown) passing through catheter body 1004 electrically connect the electrode 5002 to a RF generator and controller 6005 via a catheter hub 6000 and an electrical connector 6004.

The control algorithm for generator 6005 may detect contact with tissue by sensing impedance levels at electrode 5002. The algorithm energizes electrode 5002 when the treatment is initiated. Control of electrode 5002 may be accomplished by modulating a time and/or level of powering in accordance with the control algorithm and feedback sensed at electrode 5002 and/or temperature sensors. The algorithm may use any of temperature, voltage, current, and impedance, or any combination thereof, as control variables in the algorithm. The application of energy during the course of a bipolar RF treatment ranges from approximately 0.25 W to approximately 25 W of power for a total treatment time from approximately 10 seconds to approximately 2 minutes. The application of monopolar RF energy during the course of a treatment ranges from approximately 0.25 W to approximately 100 W of power for a total treatment time of up to approximately 5 minutes. During the application of energy, the control algorithm senses whether the control variables are within defined limits according to the software program and feedback. When a variable is outside of its limits, the energy applied to electrode 5002 is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

In another embodiment, the energy delivery surface 5002 on probe body 5001 is an ultrasound transducer controlled in a modulated fashion to maintain a treatment temperature in accordance with a control algorithm. The ultrasound transducer 5002 may produce focused or unfocused ultrasound.

Referring again to each of the FIGS. 1-8, generator 6005 is configured as a cryogenic energy source. Control of energy delivery surfaces may be accomplished by modulating a time and/or level of cryogenic powering in accordance with the generator 6005 control algorithm and feedback sensed at energy delivery surfaces and/or temperature sensors. The algorithm may use any of temperature, voltage, current, and impedance, cryogen flow rate, cryogen flow time, or any combination thereof, as control variables in the algorithm. The application of energy during the course of a treatment is based on the thermal properties of the specific cryogen being used, any of the now known cryogens for use in AF therapies being suitable, for a total treatment time from approximately 10 seconds or more. In cryogenic embodiments of the present invention, tissue treatment temperatures are below 0C (as opposed to approximately 50C to approximately 80C in non-cryogenic embodiments). During the application of energy, the control algorithm senses whether the control variables are within defined limits according to the software program and feedback. When a variable is outside of its limits, the energy applied to energy delivery surfaces is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

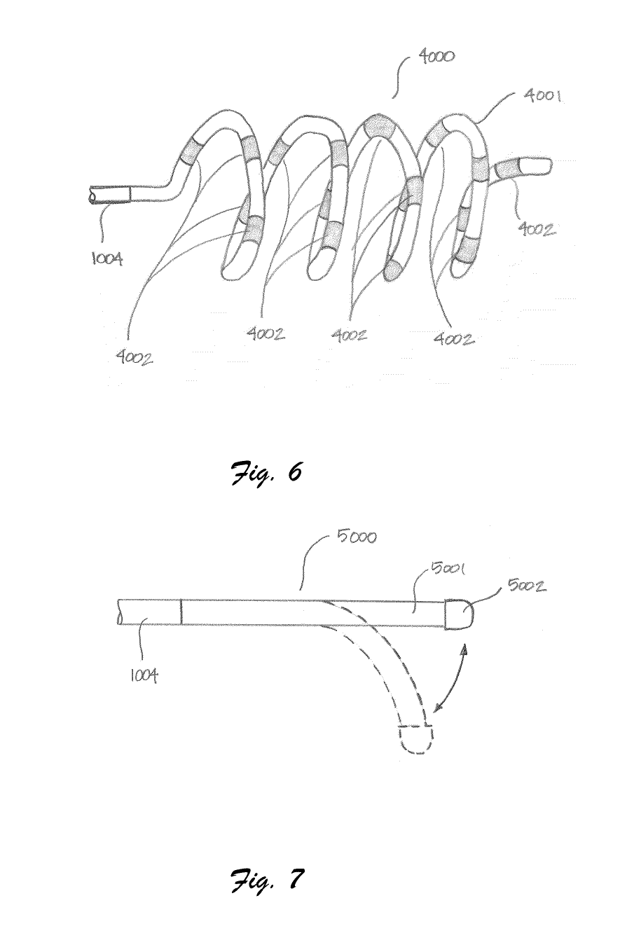

For example, the balloon 1001 of FIG. 2 may be configured to have energy delivery surfaces 1002 operatively coupled to generator 6005, which supplies cryogenic energy. In an alternate example, probe structure 5000 of FIG. 7 may be configured to have energy delivery surfaces 5002 operatively coupled to generator 6005, which supplies cryogenic energy.

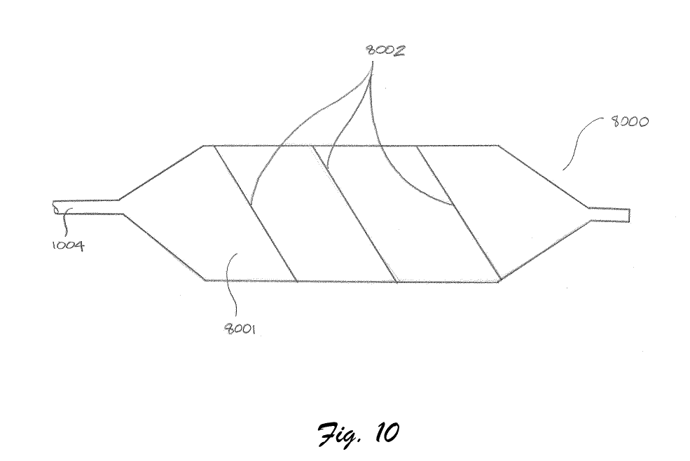

Referring to FIG. 10, an example of a cryogenic balloon structure 8000 is shown. An expandable and collapsible balloon 8001 is located at the distal end of catheter body 1004 with one or more cryogenic energy delivery surfaces 8002. The cryogenic energy delivery surfaces 8002 may be positioned either on the outer surface or the inner surface of balloon 8001. The cryogenic energy delivery surfaces 8002 are tubular in nature so as to conduct the cryogen through a fluid transmitting lumen, with a hypotube construction being an example of a cryogenic energy delivery surface 8002. Optionally, portions of the energy delivery surfaces 8002 may be insulated to allow for focused delivery of treatment energy at lesion locations in a pattern of point locations that loosely approximate a helical pattern.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims. Other embodiments are within the following claims.

1. A method for isolating a pulmonary vein for the treatment of atrial fibrillation, the method comprising:
(a) accessing the pulmonary vein with a distal portion of a catheter-based device by using an interventional technique;
(b) deploying a structure at the distal end of the catheter, comprised to include a plurality of energy delivery surfaces, such that at least one energy delivery surface is in contact with the tissue of the pulmonary vein;
(c) applying a denervating energy treatment to the tissue of the wall of the pulmonary vein adjacent the energy delivery surfaces in contact with the pulmonary vein;
(d) modulating the denervating energy treatment so as to avoid charring or vaporizing of tissue by maintaining a temperature from approximately 50C to approximately 80C adjacent an energy delivery surface during the period which energy is provided to the energy delivery surface;
(e) forming a pattern of discontinuous lesions approximating the shape of a helix, wherein individual lesions are positioned to be approximately continuous about the circumference of the pulmonary vein when viewed from a plane perpendicular to the length of the pulmonary vein and positioned to be circumferentially and axially offset from one another when viewed along the length of the pulmonary vein, and wherein the pattern of lesions isolates the pulmonary vein in an atrial fibrillation treatment.

2. The method of claim 1, wherein the catheter-based device is operatively coupled to a system further comprised of an integrated generator and controller, wherein the generator and controller modulates the energy delivery surfaces using a software-based algorithm.

3. The method of claim 1, wherein sensed feedback is used to provide input for a denervating energy delivery surface modulation calculation.

4. The method of claim 3, wherein the sensed feedback includes one or more of temperature, voltage, current, impedance.

5. The method of claim 1, wherein treatment time ranges from about 10 seconds to about 5 minutes.

6. The method of claim 1, wherein treatment power ranges from about 0.25 Watts to about 100 Watts.

7. The method of claim 1, wherein the structure at the distal end of the catheter is an inflatable and collapsible balloon comprised to include one or more energy delivery surfaces thereon, the energy delivery surfaces being circumferentially and axially offset from one another.

8. The method of claim 7, wherein the energy delivery surfaces are radiofrequency electrodes having a flexible circuit construction.

9. The method of claim 8, wherein the electrodes deliver bipolar radiofrequency energy.

10. The method of claim 8, wherein the electrodes deliver monopolar radiofrequency energy.

11. The method of claim 7, wherein the energy delivery surfaces are ultrasound transducers.

12. The method of claim 11, wherein the ultrasound transducers deliver focused ultrasound energy.

13. The method of claim 11, wherein the ultrasound transducers deliver unfocused ultrasound energy.

14. The method of claim 7, wherein the balloon diameter is between about 8 mm and about 16 mm.

15. The method of claim 14, wherein the balloon diameter tapers from its proximal end to its distal end.

16. The method of claim 7, wherein the balloon is further comprised to include one or more temperature sensors.

17. The method of claim 1, wherein the structure at the distal end of the catheter is an expandable and collapsible basket comprised to include one or more energy delivery surfaces thereon, the energy delivery surfaces being circumferentially and axially offset from one another.

18. The method of claim 17, wherein the distal end of the basket is open-ended.

19. The method of claim 17, wherein the distal end of the basket is closed-ended.

20. The method of claim 17, wherein the energy delivery surfaces are flexible radiofrequency electrodes.

21. The method of claim 20, wherein the electrodes deliver bipolar radiofrequency energy.

22. The method of claim 20, wherein the electrodes deliver monopolar radiofrequency energy.

23. The method of claim 17, wherein the energy delivery surfaces are ultrasound transducers.

24. The method of claim 23, wherein the ultrasound transducers deliver focused ultrasound energy.

25. The method of claim 23, wherein the ultrasound transducers deliver unfocused ultrasound energy.

26. The method of claim 17, wherein the basket diameter is between about 8 mm and about 16 mm.

27. The method of claim 17, wherein the basket construction is comprised of nickel-titanium.

28. The method of claim 17, wherein the balloon is further comprised to include one or more temperature sensors.

29. The method of claim 1, wherein the structure at the distal end of the catheter is an expandable and collapsible coil comprised to include one or more energy delivery surfaces thereon, the energy delivery surfaces being circumferentially and axially offset from one another along the length of the coil.

30. The method of claim 29, wherein the energy delivery surfaces are radiofrequency electrodes.

31. The method of claim 30, wherein the electrodes deliver bipolar radiofrequency energy.

32. The method of claim 30, wherein the electrodes deliver monopolar radiofrequency energy.

33. The method of claim 29, wherein the energy delivery surfaces are ultrasound transducers.

34. The method of claim 33, wherein the ultrasound transducers deliver focused ultrasound energy.

35. The method of claim 33, wherein the ultrasound transducers deliver unfocused ultrasound energy.

36. The method of claim 29, wherein the coil diameter is between about 8 mm and about 16 mm.

37. The method of claim 29, wherein the coil is further comprised to include one or more temperature sensors.

38. The method of claim 1, wherein the structure at the distal end of the catheter is a probe comprised to include one or more energy delivery surfaces thereon.

39. The method of claim 38, wherein the energy delivery surfaces are radiofrequency electrodes.

40. The method of claim 39, wherein the electrodes deliver bipolar radiofrequency energy.

41. The method of claim 39, wherein the electrodes deliver monopolar radiofrequency energy.

42. The method of claim 38, wherein the energy delivery surfaces are ultrasound transducers.

43. The method of claim 42, wherein the ultrasound transducers deliver focused ultrasound energy.

44. The method of claim 42, wherein the ultrasound transducers deliver unfocused ultrasound energy.

45. The method of claim 38, wherein the probe is configured to be deflectable to any angle up to approximately 90 degrees from the undeflected position.

46. The method of claim 38, wherein the probe is further comprised to include one or more temperature sensors.

47. A method for isolating a pulmonary vein for the treatment of atrial fibrillation, the method comprising:
(a) accessing the pulmonary vein with a distal portion of a catheter-based device by using an interventional technique;
(b) deploying a structure at the distal end of the catheter, comprised to include a plurality of energy delivery surfaces, such that at least one energy delivery surface is in contact with the tissue of the pulmonary vein;
(c) applying a cryogenic denervating energy treatment to the tissue of the wall of the pulmonary vein adjacent the energy delivery surfaces in contact with the pulmonary vein;
(d) modulating the denervating energy treatment so as to avoid damaging tissue adjacent energy delivery surfaces by maintaining a treatment temperature below 0C adjacent an energy delivery surface during the period which energy is provided to the energy delivery surface;
(e) forming a pattern of discontinuous lesions approximating the shape of a helix, wherein individual lesions are positioned to be approximately continuous about the circumference of the pulmonary vein when viewed from a plane perpendicular to the length of the pulmonary vein and positioned to be circumferentially and axially offset from one another when viewed along the length of the pulmonary vein, and wherein the pattern of lesions isolates the pulmonary vein in an atrial fibrillation treatment.

48. The method of claim 47, wherein the catheter-based device is operatively coupled to a system further comprised of an integrated generator and controller, wherein the generator and controller modulates the energy delivery surfaces using a software-based algorithm.

49. The method of claim 48, wherein sensed feedback is used to provide input for a denervating energy delivery surface modulation calculation.

50. The method of claim 49, wherein the sensed feedback includes one or more of temperature, voltage, current, impedance.

51. The method of claim 47, wherein the structure at the distal end of the catheter is an inflatable and collapsible balloon comprised to include one or more energy delivery surfaces thereon.

52. The method of claim 51, wherein the energy delivery surface is comprised of a hypotube.

53. The method of claim 52, wherein portions of the surface of the hypotube are insulated so as to focus treatment energy at the lesion locations.
